(19) United States
(12) Patent Application Publication
(10) Pub. No.: US 2003/0120430 A1
(43) Pub. Date: Jun. 26, 2003

(54) Method for producing chemical libraries enhanced with biologically active molecules

(75) Inventor: Albert Michiel van Rhee, Durham, NC (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ICAgen, Inc., Durham, NC (US)

(21) Appl. No.: 10/308,872

(22) Filed: Dec. 2, 2002

Related U.S. Application Data
(60) Provisional application No. 60/336,656, filed on Dec. 3, 2001.

(51) Int. Cl. G06F 19/00; G01N 33/48; G01N 33/50

(52) U.S. Cl. 702/19

(57) ABSTRACT

Methods and compositions for enhancing chemical libraries with biologically active molecules are taught. Relevant physicochemical descriptors that correlate with biological activity are calculated and selected. Database descriptors are identified using the physicochemical descriptors and an electronic database can be formed.

### CROSS-REFERENCE TO RELATED APPLICATIONS

This application is a non-provisional application of and claims the benefit of the filing date of U.S. Provisional Patent Application No. 60/336,656, filed on Dec. 3, 2001. This application is herein incorporated by reference for all purposes.

### BACKGROUND OF THE INVENTION

Ion channels comprise cellular proteins that regulate the flow of ions such as calcium, potassium, sodium, and chloride ions into and out of cells. They are present in all human cells and affect such processes as nerve transmission, muscle contraction and cellular secretion. Potassium ion channels, for example, are found in a variety of cells. These channels allow the flow of potassium in and/or out of the cell under certain conditions.

Numerous types of ion channel proteins are known. Some ion channels are regulated, e.g., by calcium sensitivity, voltage-gating, second messengers, extracellular ligands, and ATP-sensitivity. One type of channel protein is the voltage-gated channel protein, which is opened or closed (gated) in response to changes in electrical potential across the cell membrane. Another type of ion channel protein is a mechanically gated channel protein. In a mechanically gated channel protein, mechanical stress on the protein or a surrounding membrane opens or closes the channel. Still another type is called a ligand-gated ion channel. A ligand-gated ion channel opens or closes depending on whether a particular ligand is bound to the protein. The ligand can be either an extracellular moiety, such as a neurotransmitter, or an intracellular moiety such as an ion or nucleotide.

Ion channel modulators are potentially useful for treating disorders such as CNS (central nervous system) disorders (e.g., epilepsy), migraines, anxiety psychotic disorders such as schizophrenia, bipolar disease, and depression. They may also be useful as neuroprotective agents (e.g., to prevent stroke), for treating hyper- or hypocontractility of muscles and cardiac arrhythmias, as analgesics, and as immunosuppressants or stimulants. Because ion channel modulators have high potential therapeutic benefit, improved systems and methods for discovering ion channel modulators are desirable.

### SUMMARY OF THE INVENTION

Embodiments of the invention are directed to methods and systems of discovering pharmacologically active compounds (e.g., ion channel modulators).

One embodiment of the invention is directed to a method for creating a database system including a database of potential pharmacologically active compounds, the method comprising: a) selecting a test set of compounds; b) selecting a training set of compounds; c) entering training set data into a digital computer, wherein the training set data are derived from a biological assay on the training set of compounds; d) forming an analytical model using the training set data; e) identifying multiple physicochemical descriptors using the analytical model; f) forming a list of database descriptors using the multiple physicochemical descriptors; and g) forming a database using the database descriptors. The potential pharmacologically active compounds are preferably potential ion channel modulators.

Another embodiment of the invention is directed to a system including a database created according to the method described above.

Another embodiment of the invention is directed to a system for identifying potential ion channel modulators, comprising: a computer apparatus and a database of compounds. The database can comprise at least 100 compounds, wherein each of at least a majority of compounds in the database have at least two descriptors that characterize potential ion channel modulators.

These and other embodiments of the invention are described in further detail below with reference to the Figures.

### BRIEF DESCRIPTION OF THE DRAWINGS

FIG. 1 shows a flowchart illustrating a method according to an embodiment of the invention.

FIG. 2 shows a flowchart illustrating a process for forming an analytical model according to an embodiment of the invention.

FIG. 3 shows an example of a portion of a recursive partitioning tree.

FIG. 4 shows a system according to an embodiment of the invention.

### DETAILED DESCRIPTION

As used herein, an “ion channel modulator” is a compound that modulates the activity of an ion channel. Modulation includes, but is not limited to, the ability of a compound to increase or decrease the flow of ions through the ion channel, change ion channel open time, resting and opening threshold potential, recovery time, etc.

A “physicochemical descriptor” is any chemical and/or physical property intrinsic to a compound. Examples of physicochemical descriptors include atomic composition, molecular weight, lipophilicity, water solubility, surface polarity, ionic charge, chemical reactivity, chemical stability, hydrogen bonding potential, pKₐ, etc. Physicochemical descriptors may vary according to the compounds under investigation and may take on a range of values.

A “chemotype” is a collection of compounds that have certain “physicochemical” properties, especially those relating to molecular shape and connectivity, in common, i.e. they are homologous to some extent.

A “database descriptor” is a characteristic of a database. Multiple database descriptors can serve to define the compounds that will be included in the database. In embodiments of the invention, the database descriptor may be identified using one or more physicochemical descriptors. The physicochemical descriptors may have previously been identified from analytical models that were generated using assay data from different biological assays.

In an illustration of how a database descriptor can be formed, a physicochemical descriptor X with a range of 5 to 10 may be identified as being associated with a first ion channel modulatory activity using a first analytical model. The same physicochemical descriptor X, but with a range from 13 to 17, may be identified as being associated with a second ion channel modulatory activity using a second analytical model. The first and second analytical models may be derived using different biological assays (e.g., a first assay directed to one type of ion channel and a second assay directed to a second type of ion channel). The resulting database descriptor preferably includes a range that includes both of the ranges 5 to 10 and 13 to 17. The broader range for the database descriptor may be experimentally determined. For example, the practical range for potential ion channel modulatory activity for physicochemical descriptor X may be between 2 and 20 as determined by experimentation. The selected database descriptor may thus be X with a range from 2 to 20.

A “test library” is a collection of individual compounds. The test library may be virtual (e.g., a listing of compounds as in an electronically stored database with or without a corresponding physical collection of actual compounds) or actual (a collection of physically existing compounds). A test library may in many instances correspond to and/or define a collection of physically existing compounds so as to represent a physical library of compounds.

An “enriched library” is a collection of compounds that exhibits an increased likelihood of being ion channel modulators. The enriched library may be in the form of a database of compounds in an electronic format wherein the members have been selected to satisfy one or more database descriptors. In some embodiments, the enriched libraries will typically provide at least a 3-fold enrichment in the number of ion channel modulators as compared to the collection of compounds from which the enriched library was selected (e.g., a collection of non-prescreened compounds fabricated through a combinatorial chemistry process).

Some embodiments of the invention are directed to libraries enriched for potential pharmacologically active compounds. The compounds are preferably ion channel modulators. The electronic libraries may be in the form of a database that can be accessed by a computer apparatus such as a server computer or a client computer. Compounds in the database can be searched and/or evaluated as ion channel modulators. Compounds in the database can be selected for subsequent assaying to determine if the selected compounds are effective ion channel modulators.

Compared to a database comprising a random collection of compounds that have not previously been screened, the compounds in the database according to embodiments of the invention have a three, four, five, or more fold likelihood of being ion channel modulators. Because the compounds in the database have an increased likelihood of being effective ion channel modulators, the discovery of ion channel modulators is faster and consumes fewer resources (e.g., labor and costs) than conventional ion channel modulator discovery methods where collections of compounds have not been prescreened.

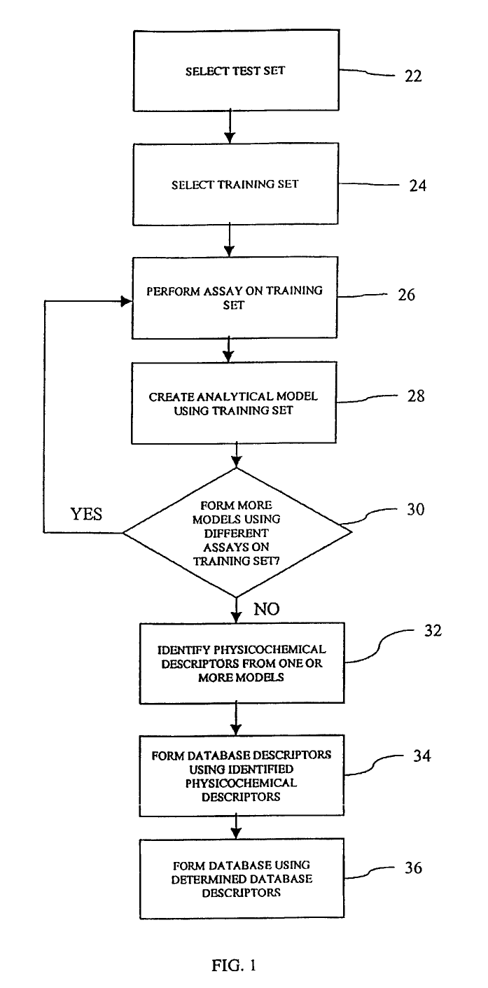

Referring to FIG. 1, in some embodiments, a test library of compounds may be selected from a larger collection of compounds. A training set of compounds is selected from the test library (step 22) and the remainder of the test library may be a test set of compounds (step 24). A biological assay may be performed on the training set to form training set data (step 26). After forming the training set data, the training set data are entered into a digital computer. An analytical model is then formed using the training set data (step 28). Additional analytical models may be formed in a similar manner to form a plurality of analytical models if desired (step 30). The different analytical models may be formed using different biological assays. Preferably, the analytical models are formed using a recursive partitioning process. Using the formed analytical models, one or more physicochemical descriptors that are associated with modulatory activity are identified (step 32). Multiple database descriptors are then identified using the identified physicochemical descriptors (step 34). Different analytical models may be formed using different assays on different ion channels. An electronic database is then formed using the multiple database descriptors (step 36).

At any point in the method, a profile may be used to screen compounds. For example, a precursor library of compounds may be screened using a profile for ion channels to create the test library of compounds. Alternatively, the profile may be used after potentially suitable compounds have been identified using one or more analytical models.

I. Pharmaceutical or Therapeutic Profile

Before or after forming the test library, some or all of the members of the compounds in the test library may be evaluated according to a predetermined pharmaceutical or a therapeutic profile. The evaluation can be conducted using, for example, Sybyl™, a commercially available molecular modeling suite of programs from Tripos, Inc., St. Louis, Mo. Using Sybyl™, 2D structural information can be transformed into 3D coordinates, and physicochemical properties based on either 2D or 3D chemical information can be obtained. 2D or 3D information can be used to determine if a compound is to be assigned a particular pharmaceutical or therapeutic profile. Using the pharmaceutical or therapeutic profile, only those compounds that fit the profile may be selected, and compounds that do not fit the profile are excluded, thus reducing the number of potential candidates. The selection of compounds using the pharmaceutical or therapeutic profile can take place before or after the analytical model is formed.

A typical pharmaceutical profile includes characteristics that make a compound desirable as a pharmaceutical agent. For example, one characteristic of a pharmaceutical profile may be the ability of a compound to dissolve in a liquid. If a compound dissolves in such liquid, then the compound fits the pharmaceutical profile. It is does not, then it does not fit the pharmaceutical profile. A typical therapeutic profile includes characteristics that make a compound desirable for a particular therapeutic purpose. For example, if the particular therapeutic purpose is to provide therapy to the brain, then the compound may have characteristics (e.g., small size) that permit it to pass the blood-brain barrier in a person. If the compound has these characteristics, then it fits the therapeutic profile. Characteristics relating to the pharmaceutical or therapeutic profile may be present in the test library and may be stored in a database along with each of the compounds in the test library. At any point, the profile information may be used to select compounds that have a higher likelihood of exhibiting a predetermined biological activity and/or are suitable for the particular pharmaceutical or therapeutic goal in mind.

An exemplary profile may be created by identifying an appropriate diversity space. Once the diversity space is identified, the profile may be created from the diversity space. The profile may be created using general scientific knowledge that is available to those of ordinary skill in the art, or could be created using past experimental results that have indicated that particular profiles are particularly useful for a given therapeutic goal.

For example, an exemplary diversity space of descriptors for ion channel modulators is shown in Table I. The diversity space may also be applicable to other protein targets. Such diversity space may be overlapping with or encompassing the diversity space for other pharmacologically and pharmaceutically active substances, such as agonists (full, partial or inverse agonists), or antagonists for cell surface receptors, G protein-coupled receptors, ion channel-coupled receptors, or nuclear receptors, or substrates or inhibitors (competitive, noncompetitive, or uncompetitive inhibitors) of enzymes affecting anabolic, metabolic, or regulatory processes.

TABLE I

| | |
|---|---|
| Pharmaceutics | |
| MW | molecular weight |
| ClogP | calculated logP, i.e. the octanol/water |
| | partitioning coefficient |
| HPSA | calculated polar surface area (see: Ertl, et |
| | al. J. Med. Chem. 43, 2000, 3714-3717) |
| FAc | calculated/estimated fraction absorbed (see: |
| | Palm, et al. J. Med. Chem. 41, 1998, 5382- |
| | 5392) |
| BBc | calculated/estimated blood-brain barrier |
| | penetration (see: Clark, D. E. J. Pharm. Sci. |
| | 88, 1999, 815-821) |
| HBCOUNT | number of hydrogen bond donors |
| NOCOUNT | total number of nitrogen and oxygen atoms |
| SULFUR | number of Sulfur atoms |
| FLUORO | number of Fluorine atoms |
| CHLORO | number of Chlorine atoms |
| BROMO | number of Bromine atoms |
| IODO | number of Iodine atoms |
| ELEMENT | number of elements other than the series: |
| | C, H, N, O, S, F, Cl, Br, I, Li, Na, K, Mg |
| ISOTOPE | number of radioisotopes, or non-natural |
| | isotopes |
| HYDROCARBON | whether or not a molecule is considered a |
| | hydrocarbon. more specifically, molecules |
| | must contain at least 1 Nitrogen atom or 1 |
| | Oxygen atom not to be considered a |
| | hydrocarbon. |
| CH2_CHAIN | length of an uninteimpted methylene chain |
| | measured in contiguous Carbon atoms |
| TERT_BUTYL_COUNT | number of t-Butyl moieties |
| DI_TERT_BUTYL | number of geminal and/or vicinal t-Butyl |
| | moieties |
| CONJUGATED_{—} | number of conjugated unsaturated bonds |
| UNSATURATED | |
| VIC_TETRAHALO | number of vicinal tetrahalogenated |
| | moieties |
| CI2 | number of CI₂ (diiodomethylene) moieties |
| DI_IODO_ARYL | number of diiodoaryl moieties |
| CYANO | number of cyano moieties |
| NITRO | number of nitro moieties |
| QUAT_NITROGEN | number of guatemary nitrogen moieties |
| OXONIUM | number of oxonium moieties |
| FURANOSE | presence or absence of furanose moieties |
| PYRANOSE | presence or absence of pyranose moieties |
| TRIPEPTIDE | number of tripeptide moieties |
| CARBOXYLATE | number of ionizable carboxylic acid |
| | moieties |
| SULFATE_SULFONATE | number of sulfate and/or sulfonate moieties |
| ESTER_COUNT | number of carboxylic ester moieties |
| POLYETHER | number of polyether moieties |
| POLYAMINE | number of polyamine moieties |
| N_OXIDE | number of N-oxide moieties |
| Potential toxicity/reactivity | |
| ACID_SULFONYL_{—} | number of acid halide and/or sulfonyl |
| HALIDE | halide moieties |
| ISO_THIO_CYANATE | number of isocyanate and/or isothiocyanate |
| | moieties |
| ALDEHYDE | number of aldehyde moieties |
| DI_M_ETHYLACETAL | number of dimethylacetal and/or |
| GEM_DI_CYANO | number of gem-dicyano moieties |
| GEM_DI_NITRO | number of gem-dinitro moieties |
| ENOL_ETHER | number of enol ether moieties |
| ENAMINE | number of enamine moieties |
| ACRYLATE | number of acrylate moieties |
| AZIRIDINE_EPOXIDE | number of aziridine and/or epoxide |
| | moieties |
| PEROXIDE | number of peroxide moieties |
| DISULFIDE | number of disulfide moieties |
| THIOL | number of thiol moieties |
| ALKYLHALIDE | number of alkylhalide moieties, i.e. the |
| | generic formula C[not aromatic](H)Hal, |
| | where Hal is either F, Cl, Br, or I |
| ARYLENEHALIDE | number of arylenehalide moieties, i.e. the |
| | generic formula C[aromatic]-C[not |
| | aromatic]Hal, where Hal is either F, Cl, Br, |
| | or I |
| AZIDE | number of azide moieties |
| HALOGENATE | number of halogenate moieties, i.e. the |
| | generic formula OHal, where Hal is either |
| | F, Cl, Br, or I |
| NITRATE_NITRITE | number of nitrate and/or nitrite moieties |
| NITRAMINE_{—} | number of nitramine and/or nitrosamine |
| NITROSAMINE | moieties |
| N_HALIDE | number of N-halide moieties, i.e. the |
| | generic formula NHal, where Hal is either |
| | F, Cl, Br, or I |
| CROWNETHER | presence or absence of crownether moieties |
| PYRROLECROWN | presence or absence of pyrrolecrown |
| | moieties |
| NITRO_ALKYL | number of nitroalkyl moieties |
| ANTHRACENE | presence or absence of anthracene moieties |
| AZO_BOND | number of azo bonds |
| TETRA_HALO_ARYL | number of tetrahaloaryl moieties |
| Generally incompatible | |
| with ion channel assays | |
| PHENALENE | number of phenalene moieties |
| STEROID | number of steroid moieties, more |
| | specifically estrogen-type steroids, |
| | androgen-type steroids, tamoxifene-like |
| | steroids, or stilbene-like steroids |
| DIHALOPHENOL | number of dihalophenol moieties, more |
| | specifically the 2,3-dihalophenol, 2,4- |
| | dihalophenol, 2,5-dihalophenol, 2,6- |
| | dihalophenol, 3,4-dihalophenol, or 3,5- |
| | dihalophenol moieties |
| CHLORAL | number of chloral chemical moieties |

The relevant pharmaceutical and therapeutic diversity space is further defined according to the criteria of Table II, which can be considered a profile for screening compounds for ion channel modulators. These criteria relate, for instance, to chemical toxicities associated with particular chemical groups, pharmacokinetic characteristics associated with particular chemical properties, chemical stability and reactivity concerns, or pharmaceutics. One or more (all or any combination) of these can be applied to a test library (or other collection of compounds) to eliminate compounds that are less likely to be ion channel modulators.

TABLE II

| | |
|---|---|
| Pharmaceutics | |
| MW | higher than 150 Dalton, but lower than |
| | 700 Dalton |
| ClogP | higher than −1, but lower than 6 |
| HPSA | higher than 0, but lower than 200 Å² |
| FAc | higher than 10% |
| BBc | depending on the therapeutic indication |
| | this value should be higher (CNS) or |
| | lower than 10% (peripheral) |
| HBCOUNT | not to exceed 6 |
| NOCOUNT | not to exceed 12 |
| SULFUR | not to exceed 2 |
| FLUORO | not to exceed 6 |
| CHLORO | not to exceed 4 |
| BROMO | not to exceed 2 |
| IODO | not to exceed 2 |
| ELEMENT | not allowed |
| ISOTOPE | for general pharmaceutical purposes: |
| | not allowed, for radiotherapy: allowed |
| HYDROCARBON | not allowed |
| CH2_CHAIN | not to exceed 6 |
| TERT_BUTYL_COUNT | not to exceed 1 |
| DI_TERT_BUTYL | not allowed |
| CONJUGATED_{—} | not to exceed 1 |
| UNSATURATED | |
| VIC_TETRAHALO | not allowed |
| CI2 | not allowed |
| DI_IODO_ARYL | not allowed |
| CYANO | not to exceed 2 |
| NITRO | not to exceed 2 |
| QUAT_NITROGEN | not to exceed 1 |
| OXONIUM | not allowed |
| FURANOSE | not allowed |
| PYRANOSE | not allowed |
| TRIPEPTIDE | not allowed |
| CARBOXYLATE | depending on the therapeutic indication |
| | this value should not exceed 1 for |
| | systemic applications and is unrestricted |
| | for topical applications |
| SULFATE_SULFONATE | depending in the therapeutic indication |
| | this value should not exceed 0 for |
| | systemic applications and is unrestricted |
| | for topical applications |
| ESTER_COUNT | not to exceed 2 |
| POLYETHER | not allowed |
| POLYAMINE | not allowed |
| N_OXIDE | not to exceed 1 |
| Potential toxicity/reactivity | |
| ACID_SULFONYL_HALIDE | not allowed |
| ISO_THIO_CYANATE | not allowed |
| ALDEHYDE | not allowed |
| DI_M_ETHYLACETAL | not allowed |
| GEM_DI_CYANO | not allowed |
| GEM_DI_NITRO | not allowed |
| ENOL_ETHER | not allowed |
| ENAMINE | not allowed |
| ACRYLATE | not allowed |
| AZIRIDINE_EPOXIDE | not allowed |
| PEROXIDE | not allowed |
| DISULFIDE | not allowed |
| THIOL | not allowed |
| ALKYLHALIDE | not allowed |
| ARYLENEHALIDE | not allowed |
| AZIDE | not allowed |
| HALOGENATE | not allowed |
| NITRATE_NITRITE | not allowed |
| NITRAMINE_NITROSAMINE | not allowed |
| N_HALIDE | not allowed |
| CROWNETHER | not allowed |
| PYRROLECROWN | not allowed |
| NITRO_ALKYL | not allowed |
| ANTHRACENE | not allowed |
| AZO_BOND | not allowed |
| TETRA_HALO_ARYL | not allowed |
| Generally incompatible with ion | |
| channel assays | |
| ALDEHYDE | not allowed |
| PHENALENE | not allowed |
| STEROID | not allowed |
| DIHALOPHENOL | not allowed |
| CHLORAL | not allowed |

II. Obtaining a Test Library of Compounds

A test library of compounds may be identified. In some embodiments, the test library has a high information content (i.e., it can be maximally diverse within the relevant pharmaceutical and/or therapeutic diversity space). The test library may contain any suitable type of compound and any suitable information that is related to the compounds. For example, the compounds in the test library may be chemical compounds or biological compounds such as polypeptides. The test library may contain data relating to the compounds in the test library. For example, each compound in the test library may have chemical data such as a hydrophobic index and a molecular weight associated with it. The test library including the compounds and the information related to the compounds may be stored in a database.

The compounds in the test library may be obtained in any suitable manner. For example, the compounds in the test library may be selected from a pre-existing set of compounds. Alternatively or additionally, the compound library may contain compounds that have been created in a synthesis process such as a combinatorial synthesis process. The test library of compounds may be synthesized either by solid or by liquid phase parallel methods known in the art. The combinatorial process can be directed by synthetic feasibility without prior knowledge of the biological target. Additionally, compounds may only exist in a virtual sense (i.e. in an electronic form stored on a hard drive or in memory in a computer), such that the compounds' characteristics can be calculated and/or predicted without the compounds being physically present. Selected candidate (second or third tier) molecules can then undergo actual synthesis and testing.

Illustratively, a new compound data set consisting of 15,000 compounds can be created using, for example, combinatorial synthesis. The new compound data set can be compared to a pre-existing data set stored in a database such as an Oracle™ relational database management system. The relational database management system may store numeric data, alphanumeric data, binary data (such as in e.g., image files), chemical data, biological activity data, analytical models, etc. Members of the new compound data set that are not redundant of the pre-existing compound data set can then be retained and added to the database containing the pre-existing compound data set. The compound data set thus defined forms the testing library.

III. Test Set and Training Set Selection

A test set of compounds and a training set of compounds are selected from the test library of compounds. Typically, the number of compounds in the training set is less than 20% of the number of compounds in the test set. After the training set is formed, the test set may be the remaining compounds in the test library. For example, a test library may contain 700,000 molecules and the formed training set may consist of 15,000 molecules. The test set may then consist of the remaining 685,000 molecules.

The information content of the training set, whether a combinatorial library candidate for HTS or a statistical analysis data set, influences the efficiency and/or utility of the analysis methodology. For this reason different experimental design strategies have been developed for diverse compound selection from a larger chemical library or chemical diversity space. (Hassan, M. et al., Mol. Diversity, 2:64-74 (1996); Higgs, R. E. et al., J. Chem. Inf. Comput. Sci., 37:861-870 (1997)).

In some embodiments, a diverse selection (DS) process can be performed using a D-optimal design strategy (Euclidian distance metric, Tanimoto Similarity Coefficient, 10,000 Monte Carlo Steps at 300 K, with a Monte Carlo Seed of 11122, and termination after 1,000 idle steps), as implemented in Cerius²™ (version 4.0; Molecular Simulations Inc., San Diego, Calif.). In a DS process, compounds are selected to maximize representation in the test library. For example, if the compounds have characteristics that make them cluster in some way (e.g., by similar morphology), then fewer compounds in the cluster are selected in order to increase the representation of other compounds in the training set.

In other embodiments, a diverse selection of 5,000 compounds was randomized with regard to the biological activity, yielding a diverse/randomized (DR) training set. The compounds in the diverse/randomized (DR) training set are randomly assigned biological activities, and a model is created. If the created model does not perform well, then the selected training set is desirable since the biological activities were randomly assigned and were not derived from actual testing. For example, 10 independent rounds of randomization can be performed where compounds are randomly (using a random number generator) assigned to the activity bins proportionately to their initial distribution, but without regard to their chemical structure and their measured biological activity.

In other embodiments, a random (RS) selection process can be used to form the training set. A training set formed by a random selection process is a stochastic sampling of a complete library, and therefore represents the information content in proportion to its distribution in the test library. In a sense, the information content is lower in a training set formed by random selection than by diverse selection. In a random selection process, densely populated areas with repetitive information are sampled more frequently than sparsely populated areas containing unique information.

IV. Assaying

The compounds in the training set may be assayed to determine their biological activity. In some embodiments, an ion channel assay may constitute a homomultimeric, or heteromultimeric isoform of a single ion channel, or multiple ion channels related through their gene sequence (i.e., a “gene family”). If an assay constituting a homomultimeric or heteromultimeric ion channel of the same gene family is used, it is possible to establish a “gene family library space” by intersecting the screening results for different ion channel types (i.e., intersecting models). A “gene family library space” refers to a library consisting of compounds that work against more than one type of ion channel. For example, compounds in a gene family library space may work against two or more types of ion channels. A “gene specific library space” may be formed by subtracting the results of different screening results for different ion channel types (i.e., differentiating models). A “gene specific library space” refers to a library consisting of compounds that work preferentially against one type of ion channel. In embodiments of the invention, such gene family libraries and gene specific libraries may be present in electronic databases.

The biological activities determined by the assaying process may be defined by two or more classes (e.g., high activity and low activity). Preferably, the biological activities may be defined by three of more related classes (e.g., high activity, moderate activity, and low activity). For example, the screening assay determines the biological activity of each compound. Each compound is then assigned to a particular class with a predetermined activity range, based on the determined biological activity. In some embodiments, the activity ranges for the different classes may include “high activity”, “moderate activity”, “low activity”, and “inactive”. The skilled artisan can determine the quantitative bounds of the classes.

Surprisingly and unexpectedly, improved predictability can be obtained by classifying activity data into more than two classes of biological activity. As shown in the Examples below, embodiments of the invention exhibit significantly improved predictability in comparison to, for example, conventional binary recursive partitioning processes. Embodiments of the invention represent an improvement over the methods published by Gao and Bajorath, Mol. Diversity, 4:115-130 (1999) (discussed below).

Any suitable assay known in the art may be used to determine the biological activity of the compounds in the test library. For example, the biological activity of the compounds may be determined using a high-throughput whole cell-based assay.

In preferred embodiments, the assay determines the ability of the compounds in the test set to modulate the activity of ion channels and the degree of activity. For example, the activity of an ion channel can be assessed using a variety of in vitro and in vivo assays, e.g., measuring current, measuring membrane potential, measuring ligand binding, measuring ion flux, e.g., potassium, or rubidium, measuring ion concentration, measuring second messengers and transcription levels, using potassium-dependent yeast growth assays, and using, e.g., voltage-sensitive dyes, ion-concentration sensitive dyes such as potassium sensitive dyes, radioactive tracers, and electrophysiology. In a specific example, changes in ion flux may be assessed by determining changes in polarization (i.e., electrical potential) of the cell or membrane expressing the potassium channel. A preferred means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595 (1997)). Whole cell currents are conveniently determined using the standard methodology (see, e.g., Hamil et al., Pflügers Archiv. 391:85 (1981)).

In an illustrative assay for a potassium channel, samples that are treated with potential potassium channel modulators are compared to control samples without the potential modulators, to examine the extent of modulation. Control samples (untreated with activators or inhibitors) are assigned a relative potassium channel activity value of 100. Modulation is achieved when the potassium channel activity value relative to the control is distinguishable from the control. The degree of activity relative to the control is generally defined in terms of the number of standard deviations from the mean. For instance, if the mean is 0%, and the 30 standard deviation is 25%, then the activity ranges could be defined as 1) 0-25%, i.e. within 1 standard deviation of the mean, 2) 25-50%, i.e. within 2 standard deviations from the mean, 3) 50-75%, i.e. within 3 standard deviations from the mean, and 4) 75-100%, i.e. within 4 standard deviations from the mean. These ranges of activity may correspond to, for example, inactive, weakly active, moderately active, and highly active, respectively.

V. Forming Analytical Models

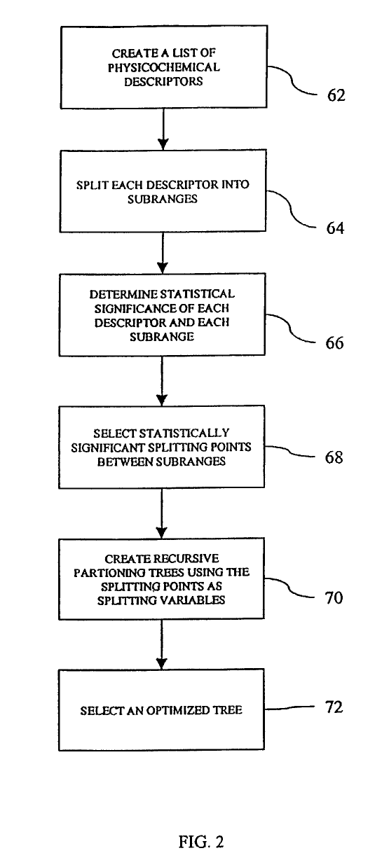

Referring to FIG. 2, a list of physicochemical descriptors is created to form a descriptor space (step 62). A physicochemical descriptor may be binary in nature, i.e. it can denote the presence or absence of a feature but not its extent. For example, a physicochemical descriptor named “heterocyclic” may denote the presence (1) or absence (0) of heteroatoms in a ring otherwise constituted by carbon atoms, but holds no information as to the number of heteroatoms present. Alternatively, a descriptor could be a continuous range descriptor. That is, it can denote the extent to which a particular feature is represented. For example, the molecular weight of a compound may be considered a continuous range descriptor. All molecules have a molecular weight, but the extent of the descriptor (e.g., a molecular weight as expressed in a range of Daltons) can be used to discriminate one molecule from another. Other examples of descriptors include the principal moment of inertia in a molecule's primary X-axis (PMI_X), a partial positive surface area (JURS_PPSA_{—}1), molecular density (Density), molecular flexibility index (phi), etc. In embodiments of the invention, hundreds or thousands of such descriptors can be considered when forming an analytical model.

A number of exemplary descriptors are provided in Cerius²™, commercially available from Molecular Simulations, Inc., San Diego, Calif. Cerius²™ is capable of generating descriptors such as spatial descriptors, structural descriptors, etc. for evaluation. It is also capable of creating recursive partitioning trees. It also allows for the variation of variables such as knot limit, tree depth, and splitting method. In embodiments of the invention, the tree depths of the recursive partitioning trees created are systematically varied until the optimal tree(s) are determined.

Each descriptor is subjected to a process called splitting, in which the range (highest descriptor value minus lowest descriptor value) is split into subranges (step 64). By systematically varying the splitting process, the statistical significance of each descriptor and its correlated range is determined (step 66). Splitting points are identified by systematically evaluating the subranges for the possibility to divide the compounds into statistically differentiated subsets based on their assigned category (step 68). The statistically most significant splitting point then becomes a splitting variable in the recursive partitioning tree.

Illustratively, a descriptor such as molecular weight can be optimized. Based on past experience or knowledge, it may be determined that the molecular weight of the particular modulator being sought would have a molecular weight ranging from 23 to 20,000. The range of 23-20,000 can then be split into progressively smaller subranges. The training set data are then applied to these splits to determine which subrange is the optimal range. For example, if it is discovered that out of 200 candidate compounds, 50 compounds having a molecular weight between 23-10,000 exhibit high activity and 150 compounds having a molecular weight between 10,000 and 20,000 exhibit low activity, then the range of 23-10,000 is selected as the more preferred range. Since a molecular weight of 10,000 splits the data, it is a splitting point and may be referred to as a “knot”. “Splitting points” and “knots” are used interchangeably and refer to values that are used to split a range for a descriptor. The 23-10,000 molecular weight continuous range descriptor is then used as a splitting variable at a node in a classification and regression tree. For example, the variable MW (molecular weight) could be used in two consecutive splits: MW<=10,000 and MW>23, to define the preferred range of 23-10,000 used to classify compounds in the test set. In this example, only one descriptor with two knots is described for simplicity of illustration. However, in other embodiments, the number of knots per descriptor may be 2 to 140 or more. Narrow or broad ranges for the descriptors can be evaluated for statistical significance.

For each set of assay data, a plurality of recursive partitioning trees is created (step 70). Tens or hundreds of trees may be generated in some embodiments. Each tree uses the descriptors, as calculated and optimized above, as splitting variables to form splits in the trees. Many such trees are created while varying such parameters as the knot limit, tree depth, and splitting method. Then, an optimal tree is selected (step 72) as an analytical model. The most desirable tree found is the one that differentiates the data the best according to biological activity.

In a typical recursive partitioning tree, parent nodes are split into two child nodes. A splitting variable splits the training set compounds into two statistically significant groups, and these two groups are classified into two respective child nodes. A Student's t-test may be used to determine the statistical significance of the split. In forming a tree, splitting methods such as the Gini Impurity, Twoing Rule, or the Greedy Improvement can be used to split the compounds. These methods are well known in the art and need not be described in further detail here (see: Breiman, L., Friedman, J. H., Olshen, R. A., Stone, C. J. Classification and Regression Trees, Wadsworth (1984)).

Once a best split is found, the classification and regression tree process repeats the search process for each child node, continuing recursively until further splitting is impossible or stopped. Splitting is impossible if only one case remains in a particular node or if all the cases in that node are of the same type. Alternatively, the process ends when there are either no more significant splits to be obtained, or when the minimum number of compounds per node is reached. The nodes at the bottom of a tree (i.e., where further splitting stops) are terminal nodes. Once a terminal node is found, the node is classified. The nodes can be classified by, for example, a plurality rule (i.e., the group with the greatest representation determines the class assignment). The tree may be pruned to the appropriate tree depth as defined at the outset of the process.

Sometimes, a molecule is included in a node because one of its descriptors increases the probability for it to be classified as “highly active”. If this molecule, by virtue of its measured activity, belongs to a class other than the one to which it has been assigned, then that molecule is a “false positive” within that node. This can occur with a series of similar (congeneric) compounds. Conversely, molecules may have been eliminated from a node based on dissimilarity, but should have been included. These molecules are “false negatives”. Models try to minimize both the number of false negatives and false positives.

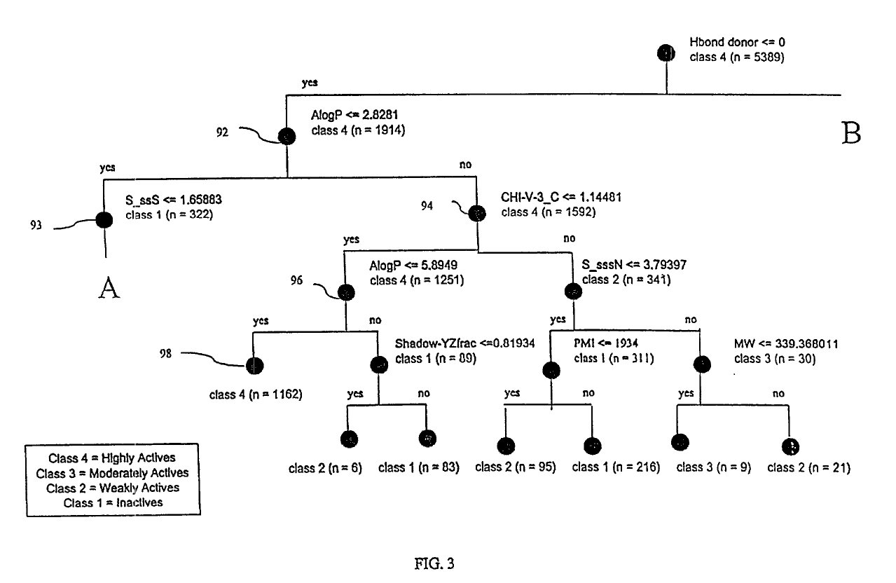

FIG. 3 shows an example of a portion of a recursive partitioning tree. The area where the letters “A” and “B” are present would have additional nodes, branches, etc. For purposes of clarity, these additional tree structures have been omitted. In this example, a node 92 may be characterized as a highly active node where the tree initially classifies 1914 members of a test set as being highly active. Then, the splitting variable “AlogP<=2.8281” may be applied to the 1914 compounds at the node 94. “AlogP” is a property of a chemical compound that is described in greater detail in Ghose A. K. and Crippen G. M. J. Comput. Chem., 7, 1986, 565. Compounds that satisfy this condition are placed in node 93 while compounds that do not are placed in node 94. The compounds assigned to these nodes 93, 94 are further split in a similar fashion, but with different rules. The classification of each node 93, 94 can be determined by determining which particular activity (i.e., highly active, moderately active, weakly active, or inactive) predominates at the node. The compounds can be split until a terminal node 98 is reached. In some embodiments, the terminal node may contain compounds, which all (or a majority of) have the same biological activity. In some instances a minority of the compounds are classified as “highly active”, but the node is statistically significantly enriched with “highly active” compounds, and therefore the entire node is deemed and labeled “highly active”. The terminal node may then be characterized by the determined biological activity. In this particular example, the nodes 92, 94, 96, 98 are all characterized as highly active nodes. The compounds classified in the terminal node 98 satisfy the following conditions:

| | |
|---|---|
| Hbond_donor <=0, yes | (“Hbond_donor” is the number of hydrogen |
| | bond donors) |
| AlogP<=2.8281, no | (“AlogP” is a calculated octanol/water |
| | partitioning coefficient) |
| CHI_V_3_{—} | (“CHI_V_3_C” is a 3rd Order Cluster |
| C <= 1.1448 1, yes | Vertex Subgraph Count Index) |
| AlogP <= 5.8949, yes | (“AlogP” is a calculated octanol/water |
| | partitioning coefficient) |

This set of physicochemical descriptors can be used to select a class of compounds that is expected to have “high biological activity” or rather a high probability of containing highly active compounds. In this example, the 1162 compounds in the terminal node 98 may serve as potential candidates for modulators. Multiple sets of physicochemical descriptors may be identified for each analytical model. Each set of physicochemical descriptors may characterize potentially highly active ion channel modulators. As will be explained in further detail below, these sets can be used to identify suitable database descriptors so that a database enriched with potential ion channel modulators can be formed.

Other details regarding the formation of analytical models are in U.S. Provisional Application No. 60/270,365 filed Feb. 20, 2000 by Michiel van Rhee et al. This application is assigned to the same assignee as the present application and is herein incorporated by reference in its entirety for all purposes.

V. Forming Database Descriptors Using Physicochemical Descriptors

As noted above, physicochemical descriptors that are characteristic of high modulation activity can be identified using one or more analytical models. A list of database descriptors can be identified using these identified physicochemical descriptors. The list of database descriptors can be used to broadly describe a larger enriched library of compounds. The database descriptors may therefore be more broadly applicable to modulators of more than one type of ion channel. In some embodiments, the list of database descriptors and their ranges may match a set of physicochemical descriptors identified from an analytical model. For example, the following may be a list of database descriptors derived from the previously mentioned set of physicochemical descriptors:

Hbond_donor<=0

AlogP>2.8281

CHI_V_{—}3_C<=1.14481

AlogP<=5.8949

In other embodiments, each database descriptor in a list may include a range that is broader than the collective ranges of similar descriptors in different sets of descriptors identified in one or more analytical models. Examples of such broad range database descriptors are provided below.

The database descriptors can be used to form a database enriched with potential ion channel modulators. The database descriptors can be used to effectively screen large compound collections. With the emergence of combinatorial chemistry, whether based on parallel, mixture, solution, or solid phase chemistry, compound libraries having vast numbers (thousands to millions) of compounds can be generated. Compounds that are evaluated for inclusion in the database may be selected from the test set, training set, test library, and/or may include compounds that are outside of the test set, training set, and/or test library.

Compounds satisfying the database descriptors can be readily identified by comparing their intrinsic physicochemical properties to the database descriptors. Compounds can be selected according to whether they satisfy any one or all of the database descriptors. For instance, each of a majority (e.g., greater than 50%) of the compounds in the database could satisfy at least two, three, or four (or more) of the database descriptors. Preferably, a vast majority (e.g., greater than 90%) of the compounds in the database satisfy at least one descriptor. For example, the italicized and bolded descriptors in Table IV below may constitute a list of database descriptors. In the electronic database that is formed, all or a vast majority (e.g., 90%) of compounds in the database preferably satisfy at least one of the italicized and bolded database descriptors in Table IV. Additionally or alternatively, at least 50%, 60%, or even 70% of the compounds in the database satisfy at least two, three or four (or more) database descriptors.

In some embodiments, databases can be formed by selecting compounds that satisfy particular sets of database descriptors. For example, Example 1 below shows nine sets of physicochemical descriptors that are descriptive of compounds that may exhibit activity towards SK3 ion channels. In this example, the physicochemical descriptors may be the same as the database descriptors. One may form a database for potential SK3 ion channel blockers by selecting compounds that satisfy each database descriptor of a set of database descriptors. For example, compounds that satisfy each descriptor in Set 1 can be included in the database. If, for example, a compound does not satisfy N_AACH<=8, then it would not satisfy Set 1 and would not be included in the database. Put another way, a database for potential SK3 ion channel blockers could be formed by selecting compounds that satisfy any of Sets 1 through 9, but satisfy each physicochemical descriptor (or database descriptor) within a given Set. Other databases could be formed in a similar manner using the information in the other Examples provided below.

An electronic database of compounds enriched for ion channel modulatory activity can be created by entering the compounds that satisfy a predetermined number and/or set of database descriptors into an electronic database. Methods of entering compound identity and physicochemical property information into a database are well known to those of ordinary skill in the art. The formed electronic database may be of any size but databases on the order of at least about 100, 500, 100,000, or 1 million are possible.

The electronic database is enriched for ion channel modulators and can improve the hit rate of primary ion channel modulator screens by at least 3-fold, thereby increasing the screening efficiency. The improved hit rate can preferably be even higher, more than 5-, 10- or 30-fold. Therefore, great efficiencies in screening are obtained (e.g., an enriched library comprising just ⅕^{th} of the test library may easily contain as much as 75% of the actives present in the test library).

VI. Using an Electronic Database for the Discovery of Ion Channel Modulators

The electronic database enriched for ion channel modulators can be used to identify effective ion channel modulators. Focusing the experimental search for ion channel modulators on compounds of the enriched library can increase the yield of active compounds identified for a given amount of experimental effort.

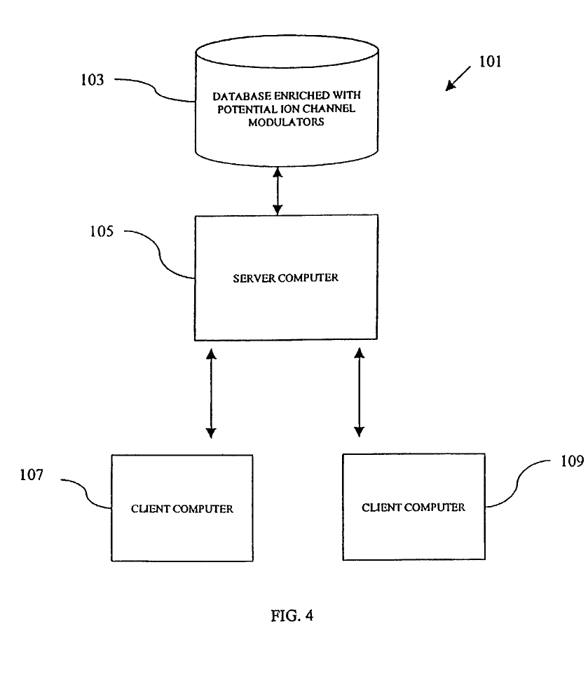

An exemplary diagram of a system according to an embodiment of the invention is shown in FIG. 4. FIG. 4 shows a system 101 including a server computer 105 in communication with a database 103. The database 103 is enriched with compounds that are ion channel modulators. The database may be stored in any suitable optical, electronic, or electro-optic computer readable information storage medium known to those of ordinary skill in the art. The server computer 105 services the requests of various client computers 107, 109.

Using the client computers 107, 109 compounds are selected from the database 103 via the server computer 105. Appropriate computer code for searching the compounds may be present on the client computers 107, 109 or the server computer 105. The compounds in the database 103 are in electronic format and can be searched. Once compounds are identified, the actual physical compounds (not shown) corresponding to the selected compounds may be obtained and assayed for their ion channel modulatory activity. As the database 103 is enriched for ion channel modulators, the likelihood of finding ion channel modulators is increased over, for example, random collections of compounds that have not been previously screened for potential ion channel modulatory activity.

In other embodiments, the server computer is not needed. For example, the database could simply reside in electronic form in a computer readable medium such as a hard disk and can be accessed by a computer apparatus. The components of the system (e.g., database, computer apparatus, etc.) may be present in the same or different housing.

### EXAMPLE

A test library of over 20,000 compounds is formed by combinatorial chemistry techniques. A training set of compounds is then selected from the test library. The training set of compounds consists of 5,000 compounds, which are selected according to D-optimal design criteria. The training set of compounds is therefore a representative sampling of the compounds present in the test library.

Prior to forming the test library, compounds are screened using the profile in Table II. Compounds that fit the profile are retained, while compounds that did not fit the profile are discarded.

The training set of compounds are assayed for: (1) the ability to block an SK3 potassium ion channel; (2) the ability to open IK1 ion channels; (3) the ability to block IK1 ion channels; (4) the ability to block PN3 ion channels; and (5) the ability to open KCNQ2/3 ion channels. From each assay, analytical models are created using the above-described recursive partitioning process. Using these analytical models, sets of physicochemical descriptors are identified (as described above). These sets are then combined to form a list of database descriptors. Further details about the specific physicochemical descriptor sets and usable assays are provided below in Exampies 1 to 5.

Table III lists 230 physicochemical descriptors that are initially selected for evaluation.

TABLE III

| Descriptor Name | Descriptor Function |
|---|---|
| S_SCH3 | S value for a single bonded methyl group |
| S_DCH2 | S value for a double bonded methylene group |
| S_SSCH2 | S value for a single/single bonded methylene group |
| S_TCH | S value for a triple bonded methyne group |
| S_DSCH | S value for a double/single bonded methyne group |
| S_AACH | S value for an aromatic/aromatic bonded methyne group |
| S_SSSCH | S value for a single/single/single bonded methyne group |
| S_DDC | S value for a double/double bonded carbon cluster |
| S_TSC | S value for a triple/single bonded carbon cluster |
| S_DSSC | S value for a double/single/single bonded carbon cluster |
| S_AASC | S value for an aromatic/aromatic/single bonded carbon cluster |
| S_AAAC | S value for an aromatic/aromatic/aromatic bonded carbon cluster |
| S_SSSSC | S value for a single/single/single/single bonded carbon cluster |
| S_SNH3 | S value for a single bonded trihydrogenanimonium group |
| S_SNH2 | S value for a sin le bonded dih dro enamino ou |
| S_SSNH2 | S value for a single/single bonded dihydrogenammonium group |
| S_DNH | S value for a double bonded monohydrogenamino group |
| S_SSNH | S value for a single/single bonded monohydrogenamino group |
| S_AANH | S value for an aromatic/aromatic bonded monohydrogenammonium |
| | group |
| S_TN | S value for a triple bonded nitrogen cluster |
| S_SSSNH | S value for a single/single/single bonded monohydrogenammonium |
| | group |
| S_DSN | S value for a double/single bonded nitrogen cluster |
| S_AAN | S value for an aromatic/aromatic bonded nitrogen cluster |
| S_SSSN | S value for a single/single/single bonded nitrogen cluster |
| S_DDSN | S value for a double/double/single bonded nitrogen cluster |
| S_AASN | S value for an aromatic/aromatic/single bonded nitrogen cluster |
| S_SSSSN | S value for a single/single/single/single bonded ammonium cluster |
| S_SOH | S value for a single bonded hydroxy group |
| S_DO | S value for a double bonded oxygen cluster |
| S_SSO | S value for a single/single bonded oxygen cluster |
| S_AAO | S value for an aromatic/aromatic oxygen cluster |
| S_SSH | S value for a single bonded sulfhydryl group |
| S_DS | S value for a double bonded sulfur cluster |
| S_SSS | S value for a single/single bonded sulfur cluster |
| S_AAS | S value for an aromatic/aromatic bonded sulfur cluster |
| S_DSSS | S value for a double/single/single bonded sulfur cluster |
| S_DDSSS | S value for a double/double/single/single bonded sulfur cluster |
| S_SPH2 | S value for a single bonded dihydrogenphosphine group |
| S_SSPH | S value for a single/single bonded monohydrogenphosphine group |
| S_DSSSP | S value for a double/single/single/single bonded phosphorous cluster |
| S_SSSSSP | S value for a single/single/single/single/single bonded phosphorous |
| | cluster |
| S_SF | S value for a single bonded fluorine cluster |
| S_SCL | S value for a single bonded chlorine cluster |
| S_SBR | S value for a single bonded bromine cluster |
| S_SI | S value for a single bonded iodine cluster |
| N_SCH3 | N value for a single bonded methyl group |
| N_DCH2 | N value for a double bonded meth lene ou |
| N_SSCH2 | N value for a single/single bonded methylene group |
| N_TCH | N value for a triple bonded methyne group |
| N_DSCH | N value for a double/single bonded methyne group |
| N_AACH | N value for an aromatic/aromatic bonded methyne group |
| N_SSSCH | N value for a single/single/single bonded methyne group |
| N_DDC | N value for a double/double bonded carbon cluster |
| N_TSC | N value for a triple/single bonded carbon cluster |
| N_DSSC | N value for a double/single/single bonded carbon cluster |
| N_AASC | N value for an aromatic/aromatic/single bonded carbon cluster |
| N_AAAC | N value for an aromatic/aromatic/aromatic bonded carbon cluster |
| N_SSSSC | N value for a single/single/single/single bonded carbon cluster |
| N_SNH3 | N value for a single bonded trihydrogenammonium group |
| N_SNH2 | N value for a single bonded dihydrogenamino group |
| N_SSNH2 | N value for a single/single bonded dihydrogenammonium group |
| N_DNH | N value for a double bonded monohydrogenamino group |
| N_SSNH | N value for a single/single bonded monohydrogenamino group |
| N_AANH | N value for an aromatic/aromatic bonded monohydrogenammonium |
| | group |
| N_TN | N value for a triple bonded nitrogen cluster |
| N_SSSNH | N value for a single/single/single bonded monohydrogenammonium |
| | group |
| N_DSN | N value for a double/single bonded nitrogen cluster |
| N_AAN | N value for an aromatic/aromatic bonded nitrogen cluster |
| N_SSSN | N value for a single/single/single bonded nitrogen cluster |
| N_DDSN | N value for a double/double/single bonded nitrogen cluster |
| N_AASN | N value for an aromatic/aromatic/single bonded nitrogen cluster |
| N_SSSSN | N value for a single/single/single/single bonded ammonium cluster |
| N_SOH | N value for a single bonded hydroxy group |
| N_DO | N value for a double bonded oxygen cluster |
| N_SSO | N value for a single/single bonded oxygen cluster |
| N_AAO | N value for an aromatic/aromatic oxygen cluster |
| N_SSH | N value for a single bonded sulfhydryl group |
| N_DS | N value for a double bonded sulfur cluster |
| N_SSS | N value for a single/single bonded sulfur cluster |
| N_AAS | N value for an aromatic/aromatic bonded sulfur cluster |
| N_DSSS | N value for a double/single/single bonded sulfur cluster |
| N_DDSSS | N value for a double/double/single/single bonded sulfur cluster |
| N_SPH2 | N value for a single bonded dihydrogenphosphine group |
| N_SSSP | N value for a single/single/single bonded phosphorous cluster |
| N_DSSSP | N value for a double/single/single/single bonded phosphorous cluster |
| N_SSSSSP | N value for a single/single/single/single/single bonded phosphorous |
| | cluster |
| N_SF | N value for a single bonded fluorine cluster |
| N_SCL | N value for a single bonded chlorine cluster |
| N_SBR | N value for a single bonded bromine cluster |
| N_SI | N value for a sin le bonded iodine cluster |
| I_SCH3 | I value for a single bonded methyl group |
| I_DCH2 | I value for a double bonded methylene group |
| I_SSCH2 | I value for a single/single bonded methylene group |
| I_TCH | I value for a triple bonded methyne group |
| I_DSCH | I value for a double/single bonded methyne group |
| I_AACH | I value for an aromatic/aromatic bonded methyne group |
| I_SSSCH | I value for a single/single/single bonded methyne group |
| I_DDC | I value for a double/double bonded carbon cluster |
| I_TSC | I value for a triple/single bonded carbon cluster |
| I_DSSC | I value for a double/single/single bonded carbon cluster |
| I_AASC | I value for an aromatic/aromatic/single bonded carbon cluster |
| I_AAAC | I value for an aromatic/aromatic/aromatic bonded carbon cluster |
| I_SSSSC | I value for a single/single/single/single bonded carbon cluster |
| I_SNH3 | I value for a single bonded trihydrogenammonium group |
| I_SNH2 | I value for a single bonded dihydrogenamino group |
| I_SSNH2 | I value for a single/single bonded dihydrogenanimonium group |
| I_DNH | I value for a double bonded monohydrogenamino group |
| I_SSNH | I value for a single/single bonded monohydrogenamino group |
| I_AANH | I value for an aromatic/aromatic bonded monohydrogenammonium |
| | group |
| I_TN | I value for a triple bonded nitrogen cluster |
| I_SSSNH | I value for a single/single/single bonded monohydrogenammonium |
| | group |
| I_DSN | I value for a double/single bonded nitrogen cluster |
| I_AAN | I value for an aromatic/aromatic bonded nitrogen cluster |
| I_SSSN | I value for a single/single/single bonded nitrogen cluster |
| I_DDSN | I value for a double/double/single bonded nitrogen cluster |
| I_AASN | I value for an aromatic/aromatic/single bonded nitrogen cluster |
| I_SSSSN | I value for a single/single/single/single bonded ammonium cluster |
| I_SOH | I value for a single bonded hydroxy group |
| I_DO | I value for a double bonded oxygen cluster |
| I_SSO | I value for a single/single bonded oxygen cluster |
| I_AAO | I value for an aromatic/aromatic oxygen cluster |
| I_SSH | I value for a single bonded sulfhydryl group |
| I_DS | I value for a double bonded sulfur cluster |
| I_SSS | I value for a single/single bonded sulfur cluster |
| I_AAS | I value for an aromatic/aromatic bonded sulfur cluster |
| I_DSSS | I value for a double/single/single bonded sulfur cluster |
| I_DDSSS | I value for a double/double/single/single bonded sulfur cluster |
| I_SPH2 | I value for a single bonded dihydrogenphosphine group |
| I_SSPH | I value for a single/single bonded monohydrogenphosphine group |
| I_SSSP | I value for a single/single/single bonded phosphorous cluster |
| I_DSSSP | I value for a double/single/single/single bonded phosphorous cluster |
| I_SSSSSP | I value for a single/single/single/single/single bonded phosphorous |
| | cluster |
| I_SF | I value for a single bonded fluorine cluster |
| I_SCL | I value for a single bonded chlorine cluster |
| I_SBR | I value for a single bonded bromine cluster |
| I_SI | I value for a single bonded iodine cluster |
| HOMO | highest occupied molecular orbital ener |
| IC | Multigraph information content index |
| BIC | Bonding information content index |
| CIC | Complementary information content index |
| SIC | Structural information content index |
| IAC_TOTAL | Information of Atomic Composition index |
| V_ADJ_MAG | Vertex Adjacency Magnitude |
| V_DIST_MAG | Vertex Distance Magnitude |
| E_ADJ_MAG | Edge Adjacency Magnitude |
| E_DIST_MAG | Edge Distance Magnitude |
| JURS_SASA | Solvent Accessible Surface Area |
| JURS_PPSA_1 | Partial Positive Surface Area |
| JURS_PNSA_1 | Partial Negative Surface Area |
| JURS_DPSA_1 | Differential Partial Charged Surface Area |
| JURS_PPSA_2 | Total Charge Weighted Positive Surface Area |
| JURS_PNSA_2 | Total Charge Weighted Negative Surface Area |
| JURS_DPSA_2 | Differential Charge Weighted Surface Area |
| JURS_PPSA_3 | Atomic Charge Weighted Positive Surface Area |
| JURS_PNSA_3 | Atomic Charge Weighted Negative Surface Area |
| JURS_DPSA_3 | Differential Atomic Charge Weigted Surface Area |
| JURS_FPSA_1 | Fractional Charged Partial Surface Area: PPSA-1/MW |
| JURS_FNSA_1 | Fractional Charged Partial Surface Area: PNSA-1/MW |
| JURS_FPSA_2 | Fractional Charged Partial Surface Area: PPSA-2/MW |
| JURS_FNSA_2 | Fractional Charged Partial Surface Area: PNSA-2/MW |
| JURS_FPSA_3 | Fractional Charged Partial Surface Area: PPSA-3/MW |
| JURS_FNSA_3 | Fractional Charged Partial Surface Area: PNSA-3/MW |
| JURS_WPSA_1 | Surface Weighted Charged Partial Surface Area: PPSA-1*SASA/1000 |
| JURS_WNSA_1 | Surface Weighted Charged Partial Surface Area: PNSA- |
| | 1*SASA/1000 |
| JURS_WPSA_2 | Surface Weighted Charged Partial Surface Area: PPSA-2*SASA/1000 |
| | 2*SASA/1000 |
| JURS_WPSA_3 | Surface Weighted Charged Partial Surface Area: PPSA-3*SASA/1000 |
| JURS_WNSA_3 | Surface Weighted Charged Partial Surface Area: PNSA- |
| | 3*SASA/1000 |
| JURS_RPCG | Relative Positive Charge |
| JURS_RNCG | Relative Negative Charge |
| JURS_RPCS | Relative Positive Charge Surface Area |
| JURS_RNCS | Relative Negative Charge Surface Area |
| JURS_TPSA | Total Polar Surface Area |
| JURS_TASA | Total Hydrophobic Surface Area |
| JURS_RPSA | Relative Polar Surface Area |
| JURS_RASA | Relative Hydrophobic Surface Area |
| SHADOW_XY | Shadow Index for the XY lane |
| SHADOW_XZ | Shadow Index for the XZ plane |
| SHADOW_YZ | Shadow Index for the YZ plane |
| SHADOW_XYFRAC | Fractional Shadow Index for the XY plane |
| SHADOW_XZFRAC | Fractional Shadow Index for the XZ plane |
| SHADOW_YZFRAC | Fractional Shadow Index for the YZ lane |
| SHADOW_NU | Ratio of largest to smallest dimension |
| SHADOW_XLENGTH | Length of the molecule in the X dimension |
| SHADOW_YLENGTH | Length of the molecule in the Y dimension |
| SHADOW_ZLENGTH | Length of the molecule in the Z dimension |
| AREA | Molecular Surface Area |
| MW | Molecular Weight |
| VM | Molecular Volume |
| DENSITY | Molecular Density |
| PMI_MAG | Principal Moment of Inertia Magnitude |
| PMI_X | Principal Moment of Inertia in the X dimension |
| PMI_Y | Principal Moment of Inertia in the Y dimension |
| PMI_Z | Principal Moment of Inertia in the Z dimension |
| ROTLBONDEDS | Number of Rotatable Bonds |
| HBOND ACCEPTOR | Number of Hydrogen Bond Acceptors |
| HBOND DONOR | Number of Hydrogen Bond Donors |
| ALOGP | calculated octanol/water partitioning coefficient |
| MOLREF | Molecular Refractivity |
| JX | Balaban Index for Relative Electronegativity |
| KAPPA_1 | Kier's First Order Shape Index |
| KAPPA_2 | Kier's Second Order Shape Index |
| KAPPA_3 | Kier's Third Order Shape Index |
| KAPPA_1_AM | Kier's Alpha-Modified First Order Shape Index |
| KAPPA_2_AM | Kier's Alpha-Modified Second Order Shape Index |
| KAPPA_3_AM | Kier's Alpha-Modified Third Order Shape Index |
| PHI | Kier & Hall's Molecular Flexibility Index |
| SC_0 | Kier & Hall's Zero Order Subgraph Count Index |
| SC_1 | Kier & Hall's First Order Subgraph Count Index |
| SC_2 | Kier & Hall's Second Order Subgraph Count Index |
| SC_3_P | Kier & Hall's Third Order Path Length Subgraph Index |
| SC_3_C | Kier & Hall's Third Order Cluster Subgraph Count Index |
| SC_3_CH | Kier & Hall's Third Order Ring and Chain Subgraph Count Index |
| CHI_0 | Kier & Hall's Zero Order Molecular Connectivity Index |
| CHI_1 | Kier & Hall's First Order Molecular Connectivity Index |
| CHI_2 | Kier & Hall's Second Order Molecular Connectivity Index |
| CHI_3_P | Kier & Hall's Third Order Path Length Molecular Connectivity Index |
| CHI_3_C | Kier & Hall's Third Order Cluster Molecular Connectivity Index |
| CHI_3_CH | Kier & Hall's Third Order Ring and Chain Molecular Connectivity |
| | Index |
| CHI_V_0 | Kier & Hall's Zero Order Vertex Subgraph Count Index |
| CHI_V_1 | Kier & Hall's First Order Vertex Subgraph Count Index |
| CHI_V_2 | Kier & Hall's Second Order Vertex Subgraph Count Index |
| CHI_V_3_P | Kier & Hall's Third Order Path Length Vertex Subgraph Index |
| CHI_V_3_C | Kier & Hall's Third Order Cluster Vertex Subgraph Count Index |
| CHI_V_3_CH | Kier & Hall's Third Order Ring and Chain Vertex Subgraph Count |
| | Index |
| WIENER | Wiener Index |
| LOG Z | Hosoya Index |
| ZAGREB | Zagreb Index |

In Table III, descriptors marked “I_”, “S_”, or “N_” (the first 138) are so-called Electrotopological descriptors. See Kier and Hall, “Molecular Structure Description”, Academic Press, New York, 1999. The “I_” designates the “intrinsic state value”, the “S_” designates the “summed differences between all intrinsic state values”, and the “N_” designates the “number of times that each intrinsic state occurs”. All hydrogen atoms are noted explicitly in the notation (group). Clusters refer to groups of atoms that are composed exclusively of heavy atoms (non-hydrogen atoms). Descriptors marked “Jurs” are defined according to Stanton and Jurs. See Stanton D. T. and Jurs P. C., Anal. Chem. 62, 1990, 2323. The AlogP is calculated according to Ghose and Crippen. See Ghose A. K. and Crippen G. M., J. Comput. Chem., 7, 1986, 565. The Kappa indices are calculated according to Hall and Kier. See: Hall L. H. and Kier L. B., J. Pharm. Sci., 67, 1978, 1743. The Balaban index is calculated according to Balaban. See: Balaban, A. T., Chem. Phys. Lett., 89(5), 1982, 399. The Wiener index is calculated according to Wiener, 1947. See: Canfield E. R., Robinson R. W., Rouvray D. H., J. Comput. Chem., 6, 1985, 598. The Hosoya index is calculated according to Hosoya, 1972. See: Hosoya H., J. Chem. Doc., 12, 1972, 181. The Zagreb index is calculated according to Bonchev, 1983. See: Bonchev D., Mekenyan O., Chem. Phys. Lett., 98, 1983, 134. Each of the above references of this paragraph and in this application are herein incorporated by reference in their entirety for all purposes.

Of the 230 physicochemical descriptors in Table III, 208 physicochemical descriptors are determined to be good candidate physicochemical descriptors. The 208 descriptors are listed in Table IV (this step can be considered an optional operation in embodiments of the invention).

All 230 physicochemical descriptors are initially considered. Those physicochemical descriptors that exhibit high variability across the test set of compounds are retained, while those that do not are removed from the analysis. In this specific example, variance/mean ratios are used to determine which physicochemical descriptors are acceptable for evaluation and which are not. The variance/mean ratios of physicochemical descriptors could be calculated for all members of a test set or all members of a test library. Other processes for screening physicochemical descriptors for analysis could alternatively be used.

Illustratively, four compounds 1 through 4 may have a physicochemical descriptor X, and the values of X may be as follows:

| Compound | value of physicochemical descriptor X |
|---|---|
| 1 | 1.2 |
| 2 | 2.4 |
| 3 | 1.4 |
| 4 | 2.2 |

The mean of the values for X is 1.8 and the variance of the X values is 0.6. The variance/mean ratio is 0.33. X can be considered an acceptable descriptor, because it exhibits different values of X that can be evaluated for statistical significance. On the other hand, the four compounds 1 through 4 may have a physicochemical descriptor Y, and the values of Y may be as follows:

| Compound | value of physicochemical descriptor Y |
|---|---|
| 1 | 2 |
| 2 | 2 |
| 3 | 2 |
| 4 | 2 |

The mean of the values for Y is 2 and the variance of Y values is 0. The variance/mean ratio is 0 and the physicochemical descriptor Y thus has low variability with respect to the set of compounds 1 to 4. Because variability in Y is low in the compound set, it is unlikely that a specific range of Y would be characteristic of high ion channel modulatory activity using the compound set. Thus, physicochemical descriptor Y may be discarded from the process of forming the database descriptors.

The specific ranges of the physicochemical descriptors in Table IV are determined using prior knowledge from past experimentation. A known set of compounds that is believed to be amenable to potential ion channel modulation was studied. The specific values for the physicochemical descriptors of the compounds of the known set are determined and broad potential useable ranges are determined for each of the 208 descriptors.

It is also possible to determine a broad range for a database descriptor by using the physicochemical descriptor ranges identified in the various analytical models that are created. For example, a range for a database descriptor X can be formed. The corresponding physicochemical descriptor X with a range of 5 to 10 may be identified as being associated with a first ion channel modulatory activity using a first analytical model. The same physicochemical descriptor X, but with a range from 13 to 17 could be identified as being associated with a second ion channel modulatory activity using a second analytical model. A range of 5 to 17 for the corresponding database descriptor X could be automatically or manually determined by taking the upper and lower bounds of the two narrower ranges identified in the analytical models.

Of the 208 descriptors in Table IV, 56 database descriptors are identified, in varying combinations, as useful in identifying ion channel modulators. These 56 database descriptors and their ranges are in italics and bolded text in Table IV. The 56 database descriptors are identified by identifying the physicochemical descriptors in Tables V-IX below (each table of physicochemical descriptors are associated with a different assay). In general, the broad ranges of the database descriptors in Table IV encompass the narrower ranges of the corresponding physicochemical descriptors determined using the various analytical models.

An electronic database is formed. Compounds that satisfy at least one of the italicized and bolded database descriptors in Table IV are included in the database. Many of the compounds satisfied at least two of the database descriptors. In this table and in other tables mentioned above, it is possible to round the values off to 1, 2, or 3 decimal places.

TABLE IV

| | Preferred Minimum | Preferred Maximum |
|---|---|---|
| Descriptor | Value | Value |
| ALOGP | −2.9883993 | 22.694191 |
| AREA | 119.033295 | 1465.38208 |
| BIC | 0 | 0.934870541 |
| CHI_0 | 4.40577745 | 65.0175781 |
| CHI_1 | 2.89384699 | 38.7669029 |
| CHI_2 | 2.06066012 | 43.0271225 |
| CHI_3_C | 0 | 15.3191242 |
| CHI_3_CH | 0 | 0.288675129 |
| CHI_3_P | 0.942809045 | 27.0375977 |
| CHI_V_0 | 3.52956867 | 56.6589203 |
| CHI_V_1 | 2.08597088 | 30.841259 |
| CHI_V_2 | 1.24005222 | 32.2471466 |
| CHI_V_3_C | 0 | 12.215168 |
| CHI_V_3_CH | 0 | 0.288675129 |
| CHI_V_3_P | 0.666447163 | 17.2236881 |
| CIC | −5.07E−07 | 4.16992521 |
| DENSITY | 0.866187715 | 2.07357904 |
| E_ADJ_MAG | 33.2192802 | 2237.95264 |
| E_DIST_MAG | 169.354904 | 98325.3906 |
| HBOND_ACCEPTOR | 0 | 33 |
| HBOND_DONOR | 0 | 10 |
| I_AAAC | 0 | 1 |
| I_AACH | 0 | 1 |
| I_AAN | 0 | 1 |
| I_AANH | 0 | 1 |
| I_AAO | 0 | 1 |
| I_AAS | 0 | 1 |
| I_AASC | 0 | 1 |
| I_AASN | 0 | 1 |
| I_DCH2 | 0 | 1 |
| I_DDSN | 0 | 1 |
| I_DDSSS | 0 | 1 |
| I_DNH | 0 | 1 |
| I_DO | 0 | 1 |
| I_DS | 0 | 1 |
| I_DSCH | 0 | 1 |
| I_DSN | 0 | 1 |
| I_DSSC | 0 | 1 |
| I_DSSS | 0 | 1 |
| I_SBR | 0 | 1 |
| I_SCH3 | 0 | 1 |
| I_SCL | 0 | 1 |
| I_SF | 0 | 1 |
| I_SI | 0 | 1 |
| I_SNH2 | 0 | 1 |
| I_SNH3 | 0 | 1 |
| I_SOH | 0 | 1 |
| I_SSCH2 | 0 | 1 |
| I_SSNH | 0 | 1 |
| I_SSNH2 | 0 | 1 |
| I_SSO | 0 | 1 |
| I_SSS | 0 | 1 |
| I_SSSCH | 0 | 1 |
| I_SSSN | 0 | 1 |
| I_SSSNH | 0 | 1 |
| I_SSSSC | 0 | 1 |
| I_SSSSN | 0 | 1 |
| I_TCH | 0 | 1 |
| I_TN | 0 | 1 |
| I_TSC | 0 | 1 |
| IAC_TOTAL | 18.1417103 | 241.612411 |
| IC | 0 | 4.75322533 |
| JURS_DPSA_1 | −761.11206 | 1031.02574 |
| JURS_DPSA_2 | 335.082857 | 43293.2425 |
| JURS_DPSA_3 | 39.9755696 | 400.62992 |
| JURS_FNSA_1 | 0.045225513 | 0.992498267 |
| JURS_FNSA_2 | −15.398263 | −0.15195901 |
| JURS_FNSA_3 | −0.45013184 | −0.01115837 |
| JURS_FPSA_1 | 0.007501733 | 0.954774487 |
| JURS_FPSA_2 | 0.108885025 | 24.9772696 |
| JURS_FPSA_3 | 0.006274459 | 0.417927185 |
| JURS_PNSA_1 | 18.8244044 | 766.908686 |
| JURS_PNSA_2 | −11898.32 | −57.154719 |
| JURS_PNSA_3 | −347.81927 | −5.4000752 |
| JURS_PPSA_1 | 5.79662899 | 1171.20505 |
| JURS_PPSA_2 | 48.234587 | 35587.5795 |
| JURS_PPSA_3 | 4.84830758 | 287.133546 |
| JURS_RASA | 0 | 1 |
| JURS_RNCG | 0.040709313 | 0.538131392 |
| JURS_RNCS | 0 | 19.0215782 |
| JURS_RPCG | 0.03070362 | 0.509361103 |
| JURS_RPCS | 0 | 64.9197629 |
| JURS_RPSA | 0 | 1 |
| JURS_SASA | 250.188157 | 1424.79863 |
| JURS_TASA | 0 | 1109.89486 |
| JURS_TPSA | 0 | 863.260306 |
| JURS_WNSA_1 | 7.08022229 | 721.96901 |
| JURS_WNSA_2 | −10979.018 | −18.472618 |
| JURS_WNSA_3 | −268.7618 | −2.6133581 |
| JURS_WPSA_1 | 4.47908603 | 1668.72708 |
| JURS_WPSA_2 | 19.7009126 | 50705.1345 |
| JURS_WPSA_3 | 2.92499331 | 366.194976 |
| JX | 0.823880792 | 6.18690634 |
| KAPPA_1 | 4.16666651 | 78.0124969 |
| KAPPA_1_AM | 3.65281558 | 74.1931305 |
| KAPPA_2 | 1.63265312 | 54.3952026 |
| KAPPA_2_AM | 1.2857542 | 50.8692741 |
| KAPPA_3 | 0.465303153 | 43.3125 |
| KAPPA_3_AM | 0.458159924 | 40.1239815 |
| LOG_Z | 0 | 15.3782053 |
| MOLREF | 22.2574978 | 342.342896 |
| MW | 85.1054 | 1177.649 |
| N_AAAC | 0 | 8 |
| N_AACH | 0 | 34 |
| N_AAN | 0 | 8 |
| N_AANH | 0 | 3 |
| N_AAO | 0 | 3 |
| N_AAS | 0 | 3 |
| N_AASC | 0 | 23 |
| N_AASN | 0 | 4 |
| N_DCH2 | 0 | 2 |
| N_DDSN | 0 | 6 |
| N_DDSSS | 0 | 4 |
| N_DNH | 0 | 2 |
| N_DO | 0 | 15 |
| N_DS | 0 | 2 |
| N_DSCH | 0 | 8 |
| N_DSN | 0 | 4 |
| N_DSSC | 0 | 10 |
| N_DSSS | 0 | 1 |
| N_SBR | 0 | 4 |
| N_SCH3 | 0 | 24 |
| N_SCL | 0 | 10 |
| N_SF | 0 | 25 |
| N_SI | 0 | 2 |
| N_SNH2 | 0 | 4 |
| N_SNH3 | 0 | 1 |
| N_SOH | 0 | 7 |
| N_SSCH2 | 0 | 44 |
| N_SSNH | 0 | 6 |
| N_SSNH2 | 0 | 1 |
| N_SSO | 0 | 8 |
| N_SSS | 0 | 8 |
| N_SSSCH | 0 | 12 |
| N_SSSN | 0 | 6 |
| N_SSSNH | 0 | 1 |
| N_SSSSC | 0 | 12 |
| N_SSSSN | 0 | 2 |
| N_TCH | 0 | 2 |
| N_TN | 0 | 4 |
| N_TSC | 0 | 4 |
| PHI | 0.782770455 | 47.1768837 |
| PMI_MAG | 42.6027485 | 16322.4655 |
| PMI_X | 11.864978 | 3940.55967 |
| PMI_Y | 23.3761312 | 11472.9547 |
| PMI_Z | 33.5823312 | 11606.5959 |
| ROTLBONDS | 0 | 62 |
| S_AAAC | −2.8028517 | 8.6260519 |
| S_AACH | −0.05010021 | 69.9859619 |
| S_AAN | 0 | 34.321331 |
| S_AANH | 0 | 8.01116753 |
| S_AAO | 0 | 15.7035122 |
| S_AAS | 0 | 4.93854427 |
| S_AASC | −63.060787 | 20.1229553 |
| S_AASN | −2.1832411 | 8.49526215 |
| S_DCH2 | 0 | 8.12057114 |
| S_DDSN | −6.303689 | 0 |
| S_DDSSS | −21.311131 | 0 |
| S_DNH | 0 | 16.2354126 |
| S_DO | 0 | 174.688416 |
| S_DS | 0 | 12.0271664 |
| S_DSCH | −0.52546287 | 13.0251637 |
| S_DSN | 0 | 17.4555016 |
| S_DSSC | −13.004069 | 7.28152037 |
| S_DSSS | −1.8727161 | 0 |
| S_SBR | 0 | 14.721714 |
| S_SCH3 | −0.39291334 | 48.5699806 |
| S_SCL | 0 | 63.2115669 |
| S_SF | 0 | 322.221619 |
| S_SI | 0 | 4.58445024 |
| S_SNH2 | 0 | 22.7867203 |
| S_SNH3 | 0 | 3.97807932 |
| S_SOH | 0 | 84.8310699 |
| S_SSCH2 | −3.9764662 | 41.2615395 |
| S_SSNH | −0.37780213 | 14.5786743 |
| S_SSNH2 | 0 | 2.33333325 |
| S_SSO | 0 | 42.7221375 |
| S_SSS | −0.43055546 | 13.6204281 |
| S_SSSCH | −10.590858 | 10.6487074 |
| S_SSSN | −0.07958579 | 14.3902235 |
| S_SSSNH | −0.98000753 | 1.4696722 |
| S_SSSSC | −93.159927 | 2.073035 |
| S_SSSSN | −0.21233392 | 2.83418369 |
| S_TCH | 0 | 10.840024 |
| S_TN | 0 | 36.372879 |
| S_TSC | 0 | 13.0166502 |
| SC_0 | 6 | 85 |
| SC_1 | 6 | 88 |
| SC_2 | 5 | 138 |
| SC_3_C | 0 | 56 |
| SC_3_CH | 0 | 1 |
| SC_3_P | 4 | 156 |
| SHADOW_NU | 1.03394026 | 7.21577532 |
| SHADOW_XLENGTH | 3.40003063 | 38.4771402 |
| SHADOW_XY | 22.9989649 | 274.825687 |
| SHADOW_XYFRAC | 0.36434914 | 0.838021779 |
| SHADOW_XZ | 7.7069402 | 172.657687 |
| SHADOW_XZFRAC | 0.45308642 | 0.836146273 |
| SHADOW_YLENGTH | 5.64638053 | 23.1956632 |
| SHADOW_YZ | 16.654245 | 162.076694 |
| SHADOW_YZFRAC | 0.462558836 | 0.838255977 |
| SHADOW_ZLENGTH | 3.40002664 | 13.2808481 |
| SIC | 0 | 1.00000012 |
| V_ADJ_MAG | 43.0195503 | 1312.85999 |
| V_DIST_MAG | 172.663849 | 91083.9063 |
| VM | 83.101518 | 1193.53548 |
| WIENER | 26 | 44514 |
| ZAGREB | 22 | 452 |

### Example 1

SK3 Ion Channel Blockers

In this example, compounds of a training set are selected and assayed for their ability to block the SK3 potassium ion channel. In an exemplary assay, changes in ion flux may be assessed by determining changes in polarization (i.e., electrical potential) of the cell or membrane expressing the potassium ion channel. In addition to those assays described above, suitable assays include: radiolabeled rubidium flux assays and fluorescence assays using voltage-sensitive dyes (see, e.g., Vestergarrd-Bogind et al., J. Membrane Biol. 88: 67-75 (1988); Daniel et al., J. Pharmacol. Meth. 25: 185-193 (1991); Holevinsky et al., J. Membrane Biology 137: 59-70 (1994)). Assays for compounds capable of inhibiting or increasing potassium flux through the channel proteins can be performed by application of the compounds to a bath solution in contact with and comprising cells having a channel of the present invention (see, e.g., Blatz et al., Nature 323: 718-720 (1986); Park, J. Physiol. 481: 555-570 (1994)). Generally, the compounds to be tested are present in the range from about 1 pM to about 100 mM, preferably from about 100 pM to about 100 μM.

Training set data are obtained after assaying. An analytical model is created using a recursive partitioning process (as described above). The nine sets of physicochemical descriptors described below are identified. The values in Table IV are the nodal values that are identified in the analytical model:

TABLE V

| | |
|---|---|
| ALOGP | 3.250900 |
| AREA | 153.716995 |
| CHI_V_0 | 15.489800 |
| CHI_V_0 | 18.481800 |
| CHI_V_3_P | 5.036920 |
| CHI_V_3_P | 5.373870 |
| CHI_V_3_P | 5.924850 |
| CIC | 0.843137 |
| HBOND_DONOR | 0 |
| IC | 3.114410 |
| IC | 3.830180 |
| IC | 4.162570 |
| JURS_DPSA_2 | 759.630005 |
| JURS_FPSA_2 | 1.675520 |
| JURS_PPSA_2 | 413.687988 |
| JURS_RPCG | 0.124410 |
| JURS_RPCS | 0.070083 |
| N_AACH | 8 |
| N_SSCH2 | 4 |
| PHI | 7.020510 |
| SC_3_C | 9 |
| S_AAN | 4.215070 |
| S_AAS | 1.028160 |
| S_DSSC | 0.787805 |
| S_SSNH | 2.921040 |
| S_SSCH2 | −0.512648 |
| S_SSSCH | −0.684882 |

| | |
|---|---|
| Set 1: | CHI_V_0 <= 18.4818 and |
| | ALOGP <= 3.2509 and |
| | CHI_V_3_P <= 5.03692 and |
| | N_AACH <= 8 and |
| | S_SSCH2 <= −0.512648 |
| Set 2: | CHI_V_0 <= 18.4818 and |
| | ALOGP <= 3.2509 and |
| | CHI_V_3_P > 5.03692 and |
| | N_SSCH2 <= 4 and |
| | JURS_DPSA_2 > 759.630005 and |
| | AREA > 153.716995 |
| Set 3: | CHI_V_0 <= 18.4818 and |
| | ALOGP <= 3.2509 and |
| | CHI_V_3_P > 5.03692 and |
| | N_SSCH2 > 4 and |
| | CHI_V_3_P < 5.37387 |
| Set 4: | CHI_V_0 <= 18.4818 and |
| | ALOGP > 3.2509 and |
| | S_AAS <= 1.02816 and |
| | S_AAN <= 4.21507 and |
| | S_SSNH <= 2.92104 and |
| | IC > 3.11441 and |
| | JURS_RPCG <= 0.12441 and |
| | CIC <= 0.843137 |
| Set 5: | CHI_V_0 <= 18.4818 and |
| | ALOGP > 3.2509 and |
| | S_AAS <= 1.02816 and |
| | S_AAN <= 4.21507 and |
| | S_SSNH <= 2.92104 and |
| | IC > 3.11441 and |
| | JURS_RPCG > 0.12441 and |
| | CHI_V_0 <= 15.4898 |
| Set 6: | CHI_V_0 <= 18.4818 and |
| | ALOGP > 3.2509 and |
| | S_AAS <= 1.02816 and |
| | S_AAN <= 4.21507 and |
| | S_SSNH > 2.92104 and |
| | PHI > 7.02051 |
| Set 7: | CHI_V_0 <= 18.4818 and |
| | ALOGP > 3.2509 and |
| | S_AAS <= 1.02816 and |
| | S_AAN > 4.21507 |
| Set 8: | CHI_V_0 > 18.4818 and |
| | SC_3_C <= 9 and |
| | JURS_FPSA_2 > 1.67552 and |
| | JURS_RPCS < 0.070083 and |
| | HBOND_DONOR <= 0 |
| Set 9: | CHI_V_0 > 18.4818 and |
| | SC_3_C > 9 and |
| | S_DSSC <= 0.787805 and |
| | CHI_V_3_P > 5.92485 and |
| | S_SSSCH <= −0.684882 and |
| | IC > 3.83018 and |
| | IC > 4.16257 |

### Example 2

IK1 Ion Channel Openers

In this example, compounds of a training set are selected and assayed for their ability to open IKI ion channels. The assays that can be used are described in U.S. Pat. No. 6,288,122. This U.S. Patent is herein incorporated by reference in its entirety and is assigned to the assignee of the present application. Training set data are obtained after assaying. An analytical model is created using a recursive partitioning process (as described above). The five sets of physicochemical descriptors described below are identified. The values in Table VII are the nodal values that were identified in the analytical model.

TABLE VI

| | |
|---|---|
| ALOGP | 3.041701 |
| DENSITY | 0.981360 |
| JURS_FNSA_2 | −1.552820 |
| JURS_RPCS | 2.320529 |
| KAPPA_3 | 1.796153 |
| MW | 532.680000 |
| SHADOW_NU | 1.847915 |
| SHADOW_XZ | 41.625555 |
| S_AAAC | 4.074209 |
| S_AACH | 22.420198 |
| S_DSSC | −1.538691 |
| S_SCL | 6.037380 |
| S_SOH | 9.169818 |

| | |
|---|---|
| Set 1: | KAPPA_3 <= 1.796153 |
| Set 2: | KAPPA_3 >= 1.796153 and |
| | S_AAAC <= 4.074209 and |
| | JURS_RPCS <= 2.320529 and |
| | SHADOW_XZ <= 41.625555 and |
| | ALOGP > 3.041701 |
| Set 3: | KAPPA_3 > 1.796153 and |
| | S_AAAC <= 4.074209 and |
| | JURS_RPCS <= 2.320529 and |
| | SHADOW_XZ > 41.625555 and |
| | DENSITY > 0.981360 and |
| | S_SCL <= 6.037380 and |
| | SHADOW_NU <= 1.847915 and |
| | S_AACH > 22.420198 |
| Set 4: | KAPPA_3 > 1.796153 and |
| | S_AAAC <= 4.074209 and |
| | JURS_RPCS <= 2.320529 and |
| | SHADOW_XZ > 41.625555 and |
| | DENSITY > 0.981360 and |
| | S_SCL > 6.037380 and |
| | S_SOH <= 9.169818 and |
| | JURS_FNS_2 <= −1.552820 and |
| | MW > 532.680000 |
| Set 5: | KAPPA_3 > 1.796153 and |
| | S_AAAC > 4.074209 |

### Example 3

IK1 Ion Channel Blockers

In this example, compounds of a training set are selected and assayed for their ability to block IK1 ion channels. The assays that that can be used are described in U.S. Pat. No. 6,288,122. This U.S. Patent is herein incorporated by reference in its entirety and is assigned to the assignee of the present application. Training set data are obtained after assaying. An analytical model is created using a recursive partitioning process (as described above). The six sets of physicochemical descriptors described below are identified. The values in Table VIII are the nodal values that are identified in the analytical model.

TABLE VII

| | |
|---|---|
| ALOGP | 3.3262 |
| ALOGP | 3.4217 |
| ALOGP | 3.9119 |
| ALOGP | 5.7487 |
| CHI_V_1 | 9.66968 |
| CHI_V_3_P | 6.51265 |
| HBOND_DONOR | 0 |
| JURS_WNSA_1 | 43.733299 |
| JURS_WNSA_2 | −44.0144 |
| KAPPA_2_AM | 7.14029 |
| MOLREF | 115.875999 |
| S_SSNH | 3.05137 |
| S_SSSN | 3.836510 |
| SC_3_C | 10 |
| SHADOW_NU | 2.40209 |
| SHADOW_YLENGTH | 8.35646 |
| WIENER | 3075 |

| | |
|---|---|
| Set 1: | HBOND_DONOR <= 0 and |
| | CHI_V_3_P <= 6.51265 and |
| | S_SSSN <= 3.83651 and |
| | JURS_WNSA_1 <= 43.733299 and |
| | ALOGP <= 3.4217 and |
| | JURS_WNSA_2 <= −44.0144 |
| Set 2: | HBOND_DONOR <= 0 and |
| | CHI_V_3_P <= 6.51265 and |
| | S_SSSN <= 3.83651 and |
| | JURS_WNSA_1 <= 43.733299 and |
| | ALOGP <= 3.4217 and |
| | JURS_WNSA_2 > −44.0144 and |
| | KAPPA_2_AM > 7.14029 |
| Set 3: | HBOND_DONOR <= 0 and |
| | CHI_V_3_P <= 6.51265 and |
| | S_SSSN <= 3.83651 and |
| | JURS_WNSA_1 <= 43.733299 and |
| | ALOGP > 3.4217 and |
| | ALOGP <= 5.7487 and |
| | SC_3_C <= 10 |
| Set 4: | HBOND_DONOR <= 0 and |
| | CHI_V_3_P <= 6.51265 and |
| | S_SSSN <= 3.83651 and |
| | JURS_WNSA_1 > 43.733299 and |
| | CHI_V_1 <= 9.66968 |
| Set 5: | HBOND_DONOR <= 0 and |
| | CHI_V_3_P <= 6.51265 and |
| | S_SSSN > 3.83651 and |
| | ALOGP > 3.9119 and |
| | SHADOW_NU <= 2.40209 |
| Set 6: | HBOND_DONOR > 0 and |
| | WIENER <= 3075 and |
| | ALOGP > 3.3262 and |
| | MOLREF <= 115.875999 and |
| | SHADOW_YLENGTH > 8.35646 |
| | and S_SSNH <= 3.05137 |

### Example 4

PN3 Ion Channel Blockers

In this example, compounds of a training set are selected and assayed for their ability to block PN3 ion channels. In an exemplary assay, the effects of the test compounds upon the function of the channels can be measured by changes in the electrical currents or ionic flux or by the consequences of changes in currents and flux. Changes in electrical current or ionic flux are measured by either increases or decreases in flux of ions such as sodium or guanidinium ions (see, e.g., Berger et al., U.S. Pat. No. 5,688,830). The cations can be measured in a variety of standard ways. They can be measured directly by concentration changes of the ions or indirectly by membrane potential or by radio-labeling of the ions.

Training set data are obtained after assaying. An analytical model is created using a recursive partitioning process (as described above). The four sets of physicochemical descriptors described below are identified. The values in Table IX are the nodal values that are identified in the analytical model.

TABLE XIII

| | |
|---|---|
| DENSITY | 1.279378 |
| JURS_DPSA_1 | −66.589728 |
| JURS_PPSA_1 | 488.419777 |
| JURS_PPSA_2 | 1404.927038 |
| N_AASC | 6 |
| PHI | 9.049939 |
| PMI_X | 443.006546 |

| | |
|---|---|
| Set 1: | PMI_X <= 443.006546 and |
| | JURS_PPSA_1 <= 488.419777 and |
| | JURS_DPSA_1 <= −66.589728 and |
| | N_AASC <= 6 and |
| | DENSITY <= 1.279378 |
| Set 2: | PMI_X <= 443.006546 and |
| | JURS_PPSA_1 <= 488.419777 and |
| | JURS_DPSA_1 <= −66.589728 and |
| | N_AASC > 6 |
| Set 3: | PMI_X > 443.006546 and |
| | JURS_PPSA_2 <= 1404.927038 |
| Set 4: | PMI_X > 443.006546 and |
| | JURS_PPSA_2 > 1404.927038 and |
| | PHI > 9.049939 |

### Example 5

KCNQ2/3 Channel Openers

In this example, compounds of a training set are selected are assayed for their ability to open KCNQ2/3 ion channels. Assays that can be used are discussed in U.S. patent application Ser. No. 09/776,791, filed Feb. 2, 2001, which is assigned to the same assignee as the present application and is herein incorporated by reference in its entirety.

Training set data are obtained after assaying. An analytical model is created using a recursive partitioning process (as described above). Eight sets of physicochemical descriptors described below are identified. The values in Table X are the nodal values that are identified in the analytical model.

TABLE IX

| | |
|---|---|
| HBOND_ACCEPTOR | 2 |
| JURS_FPSA_1 | 0.272483 |
| JURS_WPSA_1 | 142.791275 |
| S_AACH | 11.141602 |
| S_AACH | 14.666445 |
| S_AASC | 3.238945 |
| S_AASC | 5.622678 |
| S_DO | 12.777428 |
| S_DSN | 4.473095 |
| S_SCH3 | 7.741817 |
| S_SCH3 | 10.469993 |
| S_SCL | 5.875005 |
| S_SI | 2.080611 |
| S_SOH | 8.658096 |
| S_SSCH2 | 0.715278 |
| S_SSNH | 2.420389 |
| S_SSSCH | 1.733112 |
| S_TSC | 2.250016 |
| SC_3_P | 37 |
| SHADOW_ZLENGTH | 4.267653 |

| | |
|---|---|
| Set 1: | S_SSSCH <= 1.733112 and |
| | S_SSNH <= 2.420389 and |
| | JURS_FPSA_1 > 0.272483 and |
| | S_SCH3 <= 10.469993 and |
| | SHADOW_ZLENGTH > 4.267653 |
| | and S_SI > 2.080611 |
| Set 2: | S_SSSCH <= 1.733112 and |
| | S_SSNH <= 2.420389 and |
| | JURS_FPSA_1 > 0.272483 and |
| | S_SCH3 > 10.469993 |
| Set 3: | S_SSSCH <= 1.733112 and |
| | S_SSNH > 2.420389 and |
| | S_TSC <= 2.250016 and |
| | S_DSN <= 4.473095 and |
| | S_AASC <= 5.622678 and |
| | HBOND_ACCEPTOR > 2 and |
| | SC_3_P <= 37 and |
| | S_SCL <= 5.875005 and |
| | S_AASC > 3.238945 |
| Set 4: | S_SSSCH <= 1.733112 and |
| | S_SSNH > 2.420389 and |
| | S_TSC <= 2.250016 and |
| | S_DSN <= 4.473095 and |
| | S_AASC <= 5.622678 and |
| | HBOND_ACCEPTOR > 2 and |
| | SC_3_P <= 37 and |
| | S_SCL > 5.875005 and |
| | S_AACH <= 11.141602 and |
| | JURS_WPSA_1 > 142.791275 |
| Set 5: | S_SSSCH <= 1.733112 and |
| | S_SSNH > 2.420389 and |
| | S_TSC <= 2.250016 and |
| | S_DSN <= 4.473095 and |
| | S_AASC <= 5.622678 and |
| | HBOND_ACCEPTOR > 2 and |
| | SC_3_P <= 37 and |
| | S_SCL > 5.875005 and |
| | S_AACH > 11.141602 |
| Set 6: | S_SSSCH <= 1.733112 and |
| | S_SSNH > 2.420389 and |
| | S_TSC <= 2.250016 and |
| | S_DSN <= 4.473095 and |
| | S_AASC <= 5.622678 and |
| | HBOND_ACCEPTOR > 2 and |
| | SC_3_P > 37 and |
| | S_SOH <= 8.658096 and |
| | S_SCH3 > 7.741817 and |
| | S_SSCH2 <= 0.715278 |
| Set 7: | S_SSSCH = 1.733112 and |
| | S_SSNH > 2.420389 and |
| | S_TSC <= 2.250016 and |
| | S_DSN <= 4.473095 and |
| | S_AASC > 5.622678 and |
| | S_AACH <= 14.666445 |
| Set 8: | S_SSSCH > 1.733112 and |
| | S_DO > 12.777428 |

Functions such as the selection of compounds using a therapeutic or pharmaceutical profile, the creation of the analytical model (i.e., the creation of descriptors or trees, and the optimization and/or selection of models), the application of the analytical model to a test set, etc. can be performed using a digital computer that executes code embodying these and other functions. The code may be stored on any suitable computer readable media. Examples of computer readable media include magnetic, electronic, or optical disks, tapes, sticks, chips, etc. The code may also be written in any suitable computer programming language including, C, C++, etc. The digital computer used in embodiments of the invention may be a micro, mini or large frame computer using any standard or specialized operating system such as a UNIX, or Windows™ based operating system. Moreover, any suitable computer database may be used to store any data relating to the test library, test set, training set, or analytical models. Preferably, a computer database such as an Oracle™ relational database management system is used to store this information.

It is also understood that one or more steps in the method embodiments could be automatically or manually performed. For example, forming analytical models, assaying, forming database descriptors, etc. could all be automatically performed by appropriate machinery (e.g., robots, computers). Alternatively, in some embodiments, steps such as assaying, determining profiles, could be done manually while other steps (e.g., forming analytical models) could be performed automatically.

All of the references, patents, and patent applications in this application are specifically incorporated by reference for all purposes. None are admitted to be prior art with respect to the application.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed.

What is claimed is:

1. A method for creating a system including a database of potential pharmacologically active compounds, the method comprising:
a) selecting a test set of compounds;
b) selecting a training set of compounds;
c) entering training set data into a digital computer, wherein the training set data are derived from a biological assay on the training set of compounds;
d) forming an analytical model using the training set data;
e) identifying multiple physicochemical descriptors using the analytical model;
f) forming a list of database descriptors using the multiple physicochemical descriptors; and
g) forming a database using the database descriptors.

2. The method of claim 1 wherein d) comprises forming a plurality of analytical models, wherein each of the analytical models is formed using a different data set derived from a different assay and wherein e) identifying the multiple physicochemical descriptors using the analytical model includes identifying the multiple physicochemical descriptors using a plurality of analytical models.

3. The method of claim 2 wherein identifying multiple descriptors using a plurality of analytical models includes:
identifying one or more physicochemical descriptor sets associated with each analytical model within a plurality of analytical models.

4. The method of claim 3 wherein forming an electronic database using the multiple descriptors includes:
i) selecting compounds that satisfy at least one of the database descriptors; and then
ii) entering the selected compounds from i) into the database.

5. The method of claim 1 wherein forming the electronic database comprises:
i) selecting compounds that satisfy at least two of the database descriptors, and
ii) entering the selected compounds from i) into the electronic database.

6. The method of claim 1 wherein the assays are ion channel modulator screening assays.

7. The method of claim 1 wherein the analytical model is formed using a recursive partitioning process.

8. The method of claim 1 further comprising:
identifying two or more physicochemical descriptor sets associated with the analytical model, wherein the list of database descriptors comprises database descriptor sets that are the same as the two or more physicochemical descriptor sets, and
wherein forming the database using the database descriptors comprises selecting compounds that satisfy all of the database descriptors in at least one of the database descriptor sets.

9. A computer system comprising;
a computer apparatus; and
a database formed by the method according to claim 1.

10. A method for using the system of claim 9 comprising:
(a) identifying a compound in the database using the computer;
(b) physically obtaining the compound; and
(c) performing an assay on the obtained compound for ion channel modulatory activity.

11. A system for identifying potential ion channel modulators, comprising:
(a) a database of compounds comprising at least 100 compounds, wherein each of a majority of compounds in the database has at least two of the following:

| Descriptor | Minimum Value | Maximum Value |
|---|---|---|
| ALOGP | about −2.9883993 | about 22.694191 |
| AREA | about 119.033295 | about 1465.38208 |
| CHI_V_0 | about 3.52956867 | about 56.6589203 |
| CHI_V_1 | about 2.08597088 | about 30.841259 |
| CHI_V_3_P | about 0.666447163 | about 17.2236881 |
| CIC | about −5.07E−07 | about 4.16992521 |
| DENSITY | 0.866187715 | about 2.07357904 |
| HBOND_ACCEPTOR | 0 | about 33 |
| HBOND_DONOR | 0 | about 10 |
| IC | 0 | about 4.75322533 |
| JURS_DPSA_1 | about −761.11206 | about 1031.02574 |
| JURS_DPSA_2 | about 335.082857 | about 43293.2425 |
| JURS_FNSA_2 | about −15.398263 | about −0.15195901 |
| JURS_FPSA_1 | about 0.007501733 | about 0.954774487 |
| JURS_FPSA_2 | about 0.108885025 | about 24.9772696 |
| JURS_PPSA_1 | about 5.79662899 | about 1171.20205 |
| JURS_PPSA 2 | about 48.234587 | about 35587.5795 |
| JURS_RPCG | about 0.03070362 | about 0.509361103 |
| JURS_RPCS | 0 | about 64.9197629 |
| JURS_WNSA_1 | about 7.08022229 | about 721.96901 |
| JURS_WNSA_2 | about −10979.018 | about −18.472618 |
| JURS_WPSA_1 | about 4.47908603 | about 1668.72708 |
| JURS_WPSA_2 | about 19.7009126 | about 50705.1345 |
| KAPPA_2_AM | about 1.2857542 | about 50.8692741 |
| KAPPA_3 | about 0.465303153 | about 43.3125 |
| MOLREF | about 22.2574978 | about 342.342896 |
| MW | about 85.1054 | about 1177.649 |
| N_AASC | 0 | about 23 |
| N_AACH | 0 | about 34 |
| N_SSCH2 | 0 | about 44 |
| PHI | about 0.782770455 | about 47.1768837 |
| PMI_X | about 11.864978 | about 3940.55967 |
| S_AAAC | about −2.8028517 | about 8.6260519 |
| S_AACH | about −0.05010021 | about 69.9859619 |
| S_AAN | 0 | about 34.321331 |
| S_AAS | 0 | about 4.93854427 |
| S_AASC | about −63.060787 | about 20.1229553 |
| S_DO | 0 | about 174.688416 |
| S_DSN | 0 | about 17.4555016 |
| S_DSSC | about −13.004069 | about 7.28152037 |
| S_SCH3 | about −0.39291334 | about 48.5699806 |
| S_SCL | 0 | about 63.2115669 |
| S_SF | 0 | about 322.221619 |
| S_SI | 0 | about 4.58445024 |
| S_SOH | 0 | about 84.8310699 |
| S_SSCH2 | about −3.9764662 | about 41.2615395 |
| S_SSNH | about −0.37780213 | about 14.5786743 |
| S_SSSCH | about −10.590858 | about 10.6487074 |
| S_SSSN | about −0.07958579 | about 14.3902235 |
| S_TSC | 0 | |
| SC_3_C | 0 | |
| SHADOW_NU | about 1.03394026 | about 7.21577532 |
| SHADOW_XZ | about 7.7069402 | about 172.657687 |
| SHADOW_YLENGTH | about 5.64638053 | about 23.1956632 |
| SHADOW_ZLENGTH | about 3.40002664 | about 13.2808481 |
| WIENER | about 26 | about 44514 |

(b) a computer apparatus having access to the database.
